(19) United States
(12) Patent Application Publication
(10) Pub. No.: US 2015/0320961 A1
(43) Pub. Date: Nov. 12, 2015

(54) AIRWAY ACCESS DEVICE

(71) Applicant: ORIDION MEDICAL 1987 LTD., Jerusalem (IL)

(72) Inventor: Seamus Maguire, Athlone (IE)

(73) Assignee: ORIDION MEDICAL 1987 LTD., Jerusalem (IL)

(21) Appl. No.: 14/272,330

(22) Filed: May 7, 2014

(51) Int. Cl. A61M 16/08; A61M 16/10; A61M 16/00; A61M 16/04; A61B 1/267; A61B 5/097

(52) U.S. Cl. CPC A61M 16/085; A61B 1/2676; A61B 5/097; A61M 16/0003; A61M 16/0057; A61M 16/04; A61M 16/0465; A61M 16/104; A61M 16/0463; A61M 2016/003; A61M 2202/0241; A61M 2205/3334; A61M 2230/432

(57) ABSTRACT

An airway access device having a first end including a first connector configured to be connected to an airway device; a second end including a second connector configured to be connected to a ventilation machine; an airway channel extending between said first and second ends; and at least two access ports configured to allow access into a patients airway without disconnecting the ventilation machine.

### TECHNICAL FIELD

The present disclosure relates generally to the field of airway devices and breath sampling.

### BACKGROUND

When a patient is mechanically ventilated a breathing circuit is generated between airway devices, such as endotracheal tubes, endobroncheal tubes and tracheostomy tubes, and the ventilator machine. The circuit must be a closed conduit in order to ensure effective ventilation of the patient's lungs.

An increasing amount of devices are routinely used for monitoring and/or treating the ventilated patient. Each of these devices is typically supplied with their own connectors allowing them to be fitted between the breathing circuit and the airway device.

### SUMMARY

Aspects of the disclosure, in some embodiments thereof, relate to airway access devices with multiple access ports.

When airway devices, such as endotracheal tubes, brochotracheal tubes or tracheostomy tubes are inserted into a patient's airway, they serve as a conduit for passage of exhaled and inhaled gasses. Airway devices may also serve as a conduit for passage of medical instruments (e.g. a bronchoscope) into the patient's airway and lungs. However, it is often necessary to disconnect the patient from the ventilator in order to gain access into the airway device, for example for the purpose of inserting an instrument or device. Such disconnections are discouraged due to the high risk of infections and due to the reduction in effective ventilation.

The airway access devices, disclosed herein, provide a conduit (airway channel) with open ends for gas flow between the airway device and the ventilation machine. Advantageously, the airway access devices also provide at least two access ports that can be opened and closed as necessary. Beneficially, the access ports may allow access into the patient's airway whether for monitoring purposes or for the purpose of insertion of medical instruments/devices, without disconnection of the patient from ventilation.

Having two or more access ports further enables to reduce the amount of double sided connectors. Today, as clinical practice advances, an increasing amount of devices are routinely connected to a patient's breathing circuit. This consequently increases the volume of the circuit, thereby reducing the effectiveness of ventilation; increases the weight of the circuit, resulting in patient discomfort; and create opportunities for leakages. Moreover, multiple connectors connecting a variety of medical equipment may compete for the same area of connection. This often results in distancing, for example, a sampling line from the patient, consequently negatively impacting the response time of the CO₂ measurements.

Advantageously, by providing an airway access device having two or more access ports reduces the need for double ended connectors. Furthermore, the access ports may be arranged such that the overall size of the access device is minimized. In effect, a decrease in the overall weight of the breathing circuit is achieved, thereby improving patient comfort. Moreover, sampling accuracy may be refined due to avoidance of leakages, reduces dead space and reduced volume of the breathing circuit.

According to some embodiments, there is provided an airway access device having a first end including a first connector configured to be connected to an airway device; a second end including a second connector configured to be connected to a ventilation machine; an airway channel extending between the first and second ends; and at least two access ports configured to allow access into a patients airway without disconnecting the ventilation machine.

According to some embodiments, the at least two access ports include a sampling port configured to allow connection of a sampling line and a suction port configured to allow connection of a suction system and/or an insertion port configured to allow insertion of a medical instrument. According to some embodiments, the sampling line is a CO₂ sampling line. According to some embodiments, the medical instrument includes a suction catheter, a bronchoscope, a surgical tool, a tool for administration of medical agents, or any combination thereof. According to some embodiments, the insertion channel further includes a clamp configured to secure said medical instrument at an inner diameter of said airway channel. According to some embodiments, the suction system is a closed loop suction system.

According to some embodiments, the at least two access ports include a seal configured to seal of the port when not in use.

According to some embodiments, the at least two access ports include a connector and/or a channel.

According to some embodiments, the at least two access ports further include: a control port configured to allow connection of a feedback control device configured to control the operation of said ventilation machine, a sensor port configured to allow connection to a sensor, a window configured to allow observation of the airway channel or any combination thereof. Each possibility is a separate embodiment.

According to some embodiments, the feedback control device includes a flow sensor, a sampling line or both. Each possibility is a separate embodiment. According to some embodiments, the sensor includes a main stream CO₂ sensor.

According to some embodiments, the at least two access ports are located at an outer wall of the access device. According to some embodiments, the at least two access ports are mounted on, embedded in, molded on, attached to and/or an integral part of said access device

According to some embodiments, the access device includes at least three access ports.

According to some embodiments, the airway device includes an endotracheal tube, an endobroncheal tube or a tracheostomy tube. Each possibility is a separate embodiment.

According to some embodiments, there is provided an airway access system including an airway device; and an airway access device. According to some embodiments, the airway access device has a first end including a first connector configured to be connected to an airway device; a second end including a second connector configured to be connected to a ventilation machine; an airway channel extending between the first and second ends; and at least two access ports. According to some embodiments, the at least two access ports are configured to allow access into a patients airway without disconnecting the ventilation machine. According to some embodiments, the at least two access ports include a sampling port configured to allow connection of a sampling line and a suction port configured to allow connection of a suction system and/or an insertion port configured to allow insertion of a medical instrument.

According to some embodiments, the airway device includes an endotracheal tube, an endobroncheal tube or a tracheostomy tube. Each possibility is a separate embodiment.

According to some embodiments, the airway access system further includes a ventilation machine and/or an anaesthetics machine. Each possibility is a separate embodiment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 schematically illustrates an airway access device with two access ports, according to some embodiments;

FIG. 2 schematically illustrates an airway access device, with two access ports according to some embodiments;

FIG. 3 schematically illustrates an airway access device, with four access ports according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to airway access devices configured to be connected to a respiratory output device, at the one end thereof and to a ventilation machine at another end thereof. The airway device includes at least two access ports. The access ports may allow connection of additional tubes and/or devices. Additionally or alternatively, the access port may allow insertion of medical devices or tools into the patient's airways and/or lungs.

Importantly, the access device may thus eliminate the need for disconnecting the patient from the ventilation machine in order to gain access into the airway for instance when inserting a medical instrument and/or device into the airway of a patient.

According to some embodiments, there is provided an airway access device having a first end including a first connector configured to be connected to an airway device and a second end including a second connector configured to be connected to a ventilation machine. According to some embodiments, respiratory gasses flow through an airway channel formed within the access device between the first and the second end of the access device.

As used herein, the term “airway device” may refer to tubes used for ventilating patients such as but not limited to endotracheal tubes, endobroncheal tubes and tracheostomy tubes configured to be connected to a ventilation and/or an anesthetics machine.

As used herein, the term “access port” may refer to a port allowing connection and/or insertion of a medical device, instrument, tool or constituent. According to some embodiments, the access port may be located on the exterior of the access device. According to some embodiments, the access port may include a connector configured to allow connection of a medical devise (e.g. a breath sampling line). According to some embodiments, the access port is a connector configured to allow interconnection of a medical devise. As a non-limiting example, the access port may be configured to be connected to a breath sampling line (which may be further connected to capnograph). According to some embodiments, the access port may include a port/channel configured to allow insertion of a medical devise (e.g. surgical tools). According to some embodiments, the access port is a port/channel configured to allow insertion of a medical devise (e.g. surgical tools). According to some embodiments, the access port may include a window configured to allow observation into the airway channel of the access device. For example the window may allow observation of a medical device inserted into the airway channel of the access device. According to some embodiments, the access port may include an area allowing attachment of a medical device (e.g. Main stream CO₂ sensor). It is understood by one of ordinary skill in the art that each of the at least two access ports may be allocated on the access device in such manner as to minimize its overall size. In effect, this may reduce the overall weight of the breathing circuit and as a result increase patient comfort.

According to some embodiments, the access device includes at least two access ports. According to some embodiments, the term “at least two” when referring to access ports may refer to 2, 3, 4, 5, 6 or more access ports. Each possibility is a separate embodiment.

As used herein, the terms “breath sampling tube”, “sampling line” and “breath sampling line” may refer to any type of tubing(s) or any part of tubing system adapted to allow the flow of sampled breath, for example, to an analyzer, such as a capnograph. The sampling line may include tubes of various diameters, adaptors, connectors, valves, drying elements (such as filters, traps, trying tubes, such as Nafion® and the like).

As referred to herein, the terms “patient” and “subject” may interchangeably be used and may relate to a subject being connected to an airway device.

As referred to herein, the term “breathing circuit” may refer to a circuit forming a closed conduit from the airway device to the ventilation machine. The breathing circuit is typically connected to the airway device through a connector (for example a female Luer connector) and to the ventilation machine through another connector (for example a male Luer connector).

According to some embodiments, the terms “gas” and “respiratory gas” may be interchangeably used and may refer to the gasses flowing in the respiratory circuit (between the patient and the ventilation machine). According to some embodiments, the gas may be exhaled breath. According to some embodiments, the gas may be the respiratory gas supplied by the ventilation machine.

As used herein the term “located on” may refer to the access port being mounted on, embedded in, molded, attached to, being an integral part of, or otherwise positioned on the access device. Each possibility is a separate embodiment.

As used herein, the terms “proximal” and “proximal end” may refer to the part of the access device closest to the medical device, such as a ventilation machine. The length of the proximal end may for example be 0.25, 0.5, 1, 2, 3, 4, 5 cm or more. Each possibility is a separate embodiment.

As used herein, the terms “distal” and “distal end” may refer to the part of the access device closest to the airway device. The length of the distal end may for example be 0.25, 0.5, 1, 2, 3, 4, 5 cm or more. Each possibility is a separate embodiment.

As used herein, the term “certain distance” may refer to a distance larger than 5 cm, for example larger than 10 cm, 20 cm, 30 cm or 40 cm, 50 cm. Each possibility is a separate embodiment.

According to some embodiments, the access port may include a seal configured to seal of the access port when not in use. As used herein the term “seal” and “cover” may be interchangeably used and may refer to any door or lid configured to close the access port when not in use and open the access port when in use. For example the seal may seal of an insertion channel when no medical device/tool is inserted. For example the seal may seal of a connecter on the access device when no tube is connected thereto. It is understood to one of ordinary skill in the art that the seal may be made of any material impermeable to the gasses flowing in the airway channel. According to some embodiments, the seal may be configured to seal of the access port proximately to the airway channel of the access device. Alternatively, the seal may be configured to seal of the access port proximately to outer end thereof. According to some embodiments, the seal is detached, moved aside or otherwise removed when the access port is in use (e.g. when a sampling tube is connected to the access port). According to some embodiments, the seal is automatically withdrawn when the access port is in use (e.g. when a sampling tube is connected to the access port).

According to some embodiments, the at least two access ports include a sampling port configured to allow connection of sampling line and a suction port configured to allow connection to a suction system.

According to some embodiments, the at least two access ports include a sampling port configured to allow connection of a sampling line and an insertion channel.

According to some embodiments, the sampling line may be an integral part of the airway access device. According to some embodiments, the sampling line may be a separate add-on configured to be connected to the airway access device when required. According to some embodiments, the sampling line is a CO₂ sampling line.

According to some embodiments, the suction system may be an integral part of the airway access device. According to some embodiments, the suction system may be a separate add-on configured to be connected to the airway access device when required.

According to some embodiments, the sampling port is configured to sample exhaled breath from the airway device/tube (e.g. a ventilation tube).

According to some embodiments, the sampling port includes a plurality of inlets, each inlet adapted to sample breath samples from the airway channel of the access device. The inlets are physically separated from one another by a migration path, such that liquid creating a blockage in a first inlet and crossing the migration path having a surface area substantially greater than the surface area of the second inlet, and thus an increased surface tension, will be prevented from reaching the second inlet. As a result, blockage in the second inlet is prevented and breath sampling continued.

According to some embodiments, the sampling port includes a junction to which the inlets are connected through a channel, and an air collector connecting the channel with a breath sampling tube. According to some embodiments, the sampling port is positioned such that the junction is externally located to the airway access device abutting an outer wall of the access device. In another embodiment of the invention, the breath sampling port is positioned such that the junction is embedded between the outer wall and an inner wall of the airway access device. Alternatively, the junction is partially embedded in between the outer wall and inner wall.

According to some embodiments, the at least two access ports may include any of: a sampling port configured to allow connection of a sampling line, a suction port configured to allow connection to a suction system; an insertion channel, a control port configured to allow connection of a feedback control device, a sensor port configured to allow connection of a sensor, an administration port, configured to allow administration of medical agents, or any combination thereof. Each possibility is a separate embodiment.

According to some embodiments, the suction system is a closed loop suction system. According to some embodiments, the closed loop suction system includes a catheter and a sleeve enabling manipulation of the suction catheter. According to some embodiments, the sleeve and catheter are isolated. According to some embodiments, the closed suction system, may be designed to be part of a breathing circuit without disconnecting ventilator support.

According to some embodiments, the insertion channel is configured to allow insertion of a suction catheter, a bronchoscope, a surgical tool, a tool for administration of medical agents or any combination thereof. Each possibility is a separate embodiment. According to some embodiments, the insertion channel further comprises a clamp configured to secure an inserted device at an inner diameter of said airway channel. It is understood by one of ordinary skill in the art that the term “clamp”, as used herein, may refer to any element or feature suitable for retention of the inserted device at a desired position relative to the airway channel of the access device.

According to some embodiments, the feedback control device may include a flow sensor, a sampling line, or any other medical equipment used by the ventilation machine for parameter feedback control. Each possibility is separate embodiment. According to some embodiments, the feedback control device

According to some embodiments, the sensor may include a main stream CO₂ sensor or any other medical device configured to be directly connected to the access device.

According to some embodiments, there is provided an airway system including an airway device; and an airway access device(s), as disclosed herein.

According to some embodiment, the system further comprises a ventilation and/or an anaesthetics machine.

According to some embodiments, the airway device may be an endotracheal tube, an endobroncheal tube or a tracheostomy tube. Each possibility is a separate embodiment.

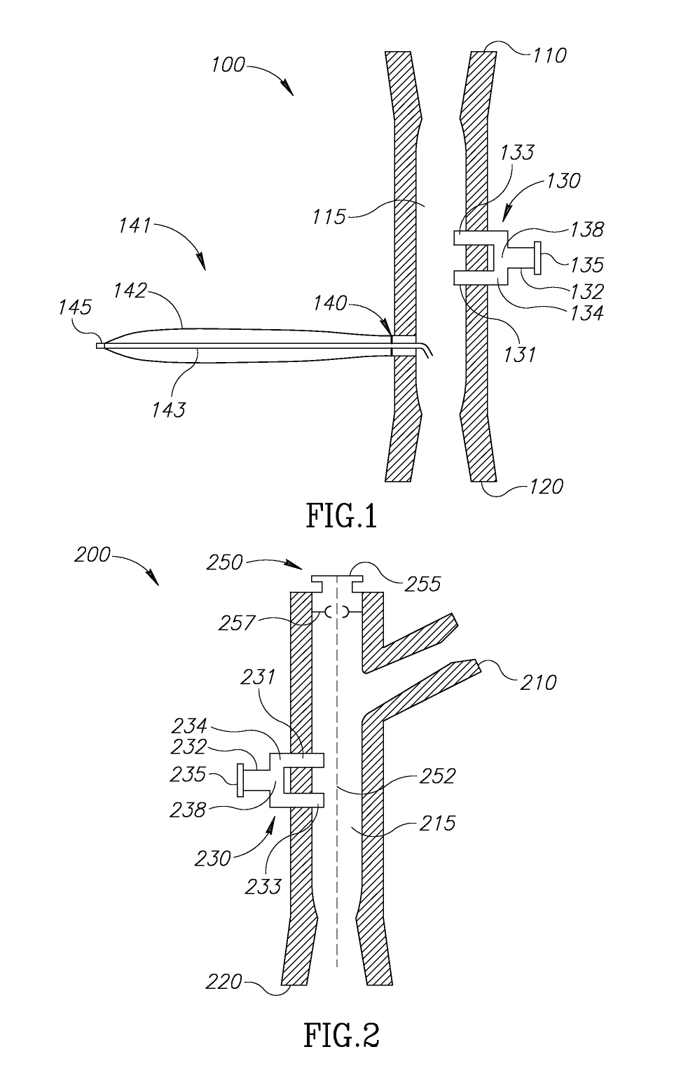

Reference is now made to FIG. 1 which schematically illustrates an airway access device 100 having two access ports, according to some embodiments. Airway access device 100 includes a first connector 110 configured to be connected to a ventilation machine (not shown) and a second connector 120 configured to be connected to an airway device, such as an endotracheal tube (not shown). An airway channel 115 extends between first connector 110 and second connector 120 allowing respiratory gasses to flow between the airway device and the ventilation machine. Airway access device 100 further includes a sampling port 130, here illustrated as including an air collector 132, a channel 134 and sampling inlets 131 and 133; however other sampling ports configured to allow sampling from airway channel 115 are also applicable and as such falls within the present disclosure. Inlets 131 and 133 are connected to each other at a junction 138. Optionally, the sampling port may have more than two inlets, for example 3, 4, 5, 6 or more inlets. Junction 138 is adapted to allow airflow between inlets 131 and 133, and channel 134 in such manner that blockage of one sampling inlet is hindered from reaching the other inlet thereby ensuring continuous breath sampling. Sampling port 130 further includes a cover 135 configured to seal off sampling port 130 when not in use. Cover 135 is further configured to be removed when a sampling tube (not shown) is connected to sampling port 130. Alternatively, the sampling tube may be permanently connected to airway access device 100 (option not shown), forming an integral part therewith. Airway access device 100 also includes a suction port 140 configured to allow connection of a closed suction system 141 including a suction catheter 143 placed within an isolating sleeve 142. Suction system 141 may include a cover 145 configured to seal of the suction catheter, when not in use. Alternatively, cover 145 may be configured to cover suction port 140 when no suction system is attached thereto. Thus, as understood by one of ordinary skill in the art, suction system 141 (or other suitable suction systems) may be an integral part of airway access device 100 or be separate add-ons configured to be connected to airway access device 100 when required.

Reference is now made to FIG. 2 which schematically illustrates an airway access device 200 having two access ports, according to some embodiments. Airway access device 200 includes a first connector 210 configured to be connected to a ventilation machine (not shown) and a second connector 220 configured to be connected to an airway device, such as an endotracheal tube (not shown). An airway channel 215 extends between first connector 210 and second connector 220 allowing respiratory gasses to flow between the airway device and the ventilation machine. Airway access device 200 further includes a sampling port 230, here illustrated as including an air collector 232, a channel 234 and sampling inlets 231 and 233; however other sampling ports configured to allow sampling from airway channel 215 are also applicable and as such falls within the present disclosure. Inlets 231 and 233 are connected to each other at a junction 238. Optionally, the sampling port may have more than two inlets, for example 3, 4, 5, 6 or more inlets. Junction 238 is adapted to allow airflow between inlets 231 and 233, and channel 234 in such manner that blockage of one sampling inlet is hindered from reaching the other inlet thereby ensuring continuous breath sampling. Sampling port 230 further includes a cover 235 configured to seal off sampling port 230 when not in use. Cover 235 is further configured to be removed when a sampling tube (not shown) is connected to sampling port 230. Alternatively, the sampling tube may be permanently connected to airway access device 200 (option not shown), forming an integral part therewith. Airway access device 200 also includes an insertion port 250 configured to allow insertion of medical instruments (e.g. a surgical tool). Airway access device 200 further includes a clamp 257 configured to restrain a medical instrument, inserted into airway access device 200 through insertion port 250. For example, clamp 257 may restrain the inserted medical equipment at an inner diameter 252 of airway channel 215. Insertion port 250 may include a cover 255 configured to seal of the insertion port, when not in use.

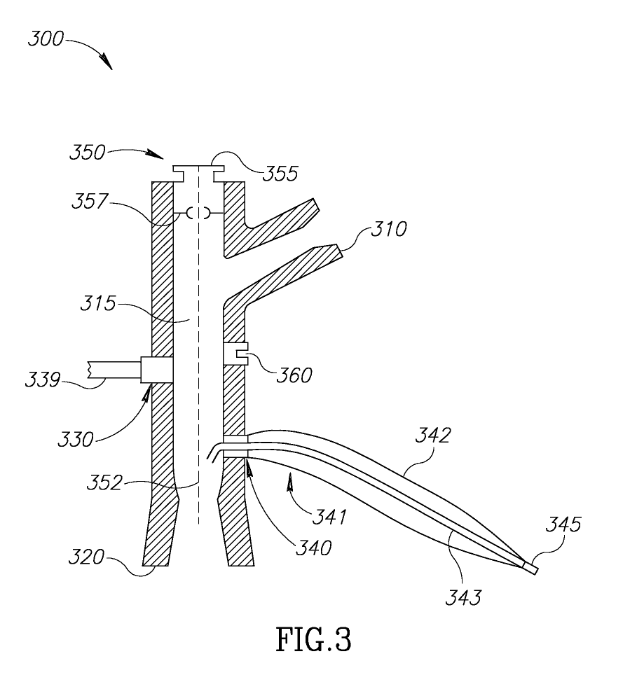

Reference is now made to FIG. 3 which schematically illustrates an airway access device 300 having multiple access ports, according to some embodiments. Airway access device 300 includes a first connector 310 configured to be connected to a ventilation machine (not shown) and a second connector 320 configured to be connected to an airway device, such as an endotracheal tube (not shown). An airway channel 315 extends between first connector 310 and second connector 320 allowing respiratory gasses to flow between the airway device and the ventilation machine. Airway access device 300 further includes a sampling port 330 configured to allow sampling from airway channel 315. Connected to sampling port 330 is a sampling line 339. Sampling line 339 may be an integral part of airway access device 300. Alternatively, sampling line 339 may be connected to sampling port 330 when required. When no sampling line is connected to sampling port 330 a cover (not shown) may cover sampling port 330 to avoid leakages therethrough. Airway access device 300 also includes a suction port 340 configured to allow connection of a closed suction system 341 including a suction catheter 343 placed within an isolating sleeve 342. Suction system 341 may include a cover 345 configured to seal of the suction catheter, when not in use. Alternatively, cover 345 may be configured to cover suction port 340 when no suction system is attached thereto. Thus, as understood by one of ordinary skill in the art, suction system 341 (or other suitable suction systems) may be an integral part of airway access device 300 or be separate add-ons configured to be connected to airway access device 300 when required. Airway access device 300 additionally includes an insertion port 350 configured to allow insertion of medical instruments (e.g. a bronchoscope). Airway access device 300 further includes a clamp 357 configured to restrain a medical instrument, inserted into airway access device 300 through insertion port 350. For example, clamp 357 may restrain the inserted medical equipment at an inner diameter 352 of airway channel 315. Insertion port 350 may include a cover 355 configured to seal of the insertion port, when not in use. Airway access device 300 also includes an additional access port, namely window 360. Window 360 may be configured to allow a clinician to observe the inside of airway channel 315, for example to ensure that an inserted tool is properly placed within airway channel 315 and/or is properly restrained by clamp 357. Alternatively, window 360 may serve as an attachment area for a monitoring device (not shown) configured to be directly connected to airway access device 300, such as, but not limited to a main stream CO₂ monitor. Airway access device 300 is here illustrated as having four access ports; however one of ordinary skill in the art will readily understand that the number of access ports may vary between different airway access devices and that the exemplified number of access ports serves an illustrative purpose only.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. An airway access device comprising:
a first end comprising a first connector configured to be connected to an airway device;
a second end comprising a second connector configured to be connected to a ventilation machine;
an airway channel extending between said first and second ends; and
at least two access ports configured to allow access into a patients airway without disconnecting said ventilation machine, said at least two access ports comprising a sampling port configured to allow connection of a sampling line and a suction port configured to allow connection of a suction system and/or an insertion port configured to allow insertion of a medical instrument.

2. The access device of claim 1, wherein said at least two access ports comprise a seal configured to seal of the port when not in use.

3. The access device of claim 1, wherein said at least two access ports comprise a connector and/or a channel.

4. The access device of claim 1, wherein said sampling line is a CO₂ sampling line.

5. The access device of claim 1, wherein said medical instrument comprises a suction catheter, a bronchoscope, a surgical tool, a tool for administration of medical agents, or any combination thereof.

6. The access device of claim 1, wherein said insertion channel further comprises a clamp configured to secure said medical instrument at an inner diameter of said airway channel.

7. The access device of claim 1, wherein said suction system is a closed loop suction system.

8. The access device of claim 1, wherein said at least two access ports further comprise: a control port configured to allow connection of a feedback control device configured to control the operation of said ventilation machine, a sensor port configured to allow connection to a sensor, a window configured to allow observation of said airway channel or any combination thereof.

9. The access device of claim 8, wherein said feedback control device comprises a flow sensor, a sampling line or both.

10. The access device of claim 8, wherein said sensor comprises a main stream CO₂ sensor.

11. The access device of claim 1, comprising at least three access ports.

12. The access device of claim 1, wherein said at least two access ports are located at an outer wall of said access device.

13. The access device of claim 1, wherein said at least two access ports are mounted on, embedded in, molded on, attached to and/or an integral part of said access device

14. The access device of claim 1, wherein said airway device comprises an endotracheal tube, an endobroncheal tube or a tracheostomy tube.

15. An airway access system comprising:
an airway device; and
an airway access device comprising a first end comprising a first connector configured to be connected to an airway device;
a second end comprising a second connector configured to be connected to a ventilation machine;
an airway channel extending between said first and second ends; and
at least two access ports configured to allow access into a patients airway without disconnecting said ventilation machine, said at least two access ports comprising a sampling port configured to allow connection of a sampling line and a suction port configured to allow connection of a suction system and/or an insertion port configured to allow insertion of a medical instrument.

16. The airway system of claim 15, wherein said airway device comprises an endotracheal tube, an endobroncheal tube or a tracheostomy tube.

17. The airway system of claim 15, further comprising a ventilation machine and/or an anaesthetics machine.
